(19) United States
(12) Patent Application Publication
(10) Pub. No.: US 2023/0285541 A1
(43) Pub. Date: Sep. 14, 2023

(54) METHODS AND COMPOSITIONS FOR INCREASING IMMUNITY AGAINST CORONAVIRUSES

(71) Applicant: LEYDEN LABORATORIES B.V., Amsterdam (NL)

(72) Inventors: Domenico VALERIO, Amsterdam (NL); Jaap GOUDSMIT, Amsterdam (NL); Stefan Frederik Franciscus VERLINDEN, Amsterdam (NL)

(21) Appl. No.: 18/005,900

(22) PCT Filed: Jul. 20, 2021
(86) PCT No.: PCT/NL2021/050461

§ 371 Date: Jan. 18, 2023

(30) Foreign Application Priority Data
Jul. 20, 2020 (NL) 2026094

(51) Int. Cl. A61K 39/215; A61K 39/39

(52) U.S. Cl. CPC A61K 39/215; A61K 39/39; A61K 2039/543

(57) ABSTRACT

The disclosure provides methods and compositions for increasing immunity against coronaviruses, in particular highly pathogenic coronaviruses. Compositions are provided comprising peptides comprising at least a part of the S2 ectodomain of the S (spike) protein from at least one human coronaviruses (HCoV) selected from HCoV-NL63, HCoV-OC43, HCoV-229E and HCoV-HKU1, as well as compositions comprising nucleic acid molecules encoding at least a part of the S2 ectodomain of the S (spike) protein from at least one human coronaviruses (HCoV) selected from HCoV-NL63, HCoV-OC43, HCoV-229E and HCoV-HKU1. Compositions described herein are particularly useful as vaccines, in particular against highly pathogenic coronaviruses such as SARS-CoV-1, MERS-CoV and/or SARS-CoV-2 as well as cross-species transmission of typically non-human coronaviruses.

### CROSS REFERENCE TO RELATED APPLICATIONS

This application is a national phase entry under 35 U.S.C. § 371 of International Patent Application PCT/NL2021/050461, filed Jul. 20, 2021, designating the United States of America and published in English as International Patent Publication WO WO2022/019758 on Jan. 27, 2022, which claims the benefit under Article 8 of the Patent Cooperation Treaty to The Netherlands Patent Application Serial No. 2026094, filed Jul. 20, 2020, the entireties of which are hereby incorporated by reference.

### FIELD OF THE INVENTION

The disclosure provides methods and compositions for increasing immunity against coronaviruses, in particular highly pathogenic coronaviruses. Compositions are provided comprising peptides comprising at least a part of the S2 ectodomain of the S (spike) protein from at least one human coronaviruses (HCoV) selected from HCoV-NL63, HCoV-OC43, HCoV-229E and HCoV-HKU1, as well as compositions comprising nucleic acid molecules encoding at least a part of the S2 ectodomain of the S (spike) protein from at least one human coronaviruses (HCoV) selected from HCoV-NL63, HCoV-OC43, HCoV-229E and HCoV-HKU1. Compositions described herein are particularly useful as vaccines, in particular against highly pathogenic coronaviruses such as SARS-CoV-1, MERS-CoV and/or SARS-CoV-2 as well as cross-species transmission of typically non-human coronaviruses.

### BACKGROUND OF THE INVENTION

Coronaviruses are enveloped RNA viruses that can infect mammals and birds. Alphacoronaviruses and betacoronaviruses infect mammals (e.g., bovine coronavirus (BCoV); canine coronavirus (CCoV), feline coronavirus (FCoV), and human coronavirus (HCoV), while gammacoronaviruses and deltacoronaviruses infect generally infect birds. Most coronaviruses infect only one host species. However, cross-species transmission can also occur and is a significant cause of disease emergence in humans (i.e., zoonosis).

Coronaviruses encode a number of viral proteins including the spike protein, membrane protein, envelope protein and the nucleocapsid protein. The spike protein (S protein) is a large type I transmembrane, class I fusion protein. The ectodomain of the S protein contains an S1 domain and an S2 domain. The N-terminal S1 domain comprises receptor binding domains (RBD) and is responsible for receptor binding. The S1 domain, in particular the 51 RBD, has been a target site of a number of antibodies and vaccines developed against specific coronaviruses. The C-terminal S2 ectodomain is responsible for fusion and comprises an UH domain (upstream helix), a fusion peptide, two heptad repeats (HR1 and HR2), a central helix, and a beta hairpin. These regions and exemplary sequences of such regions are known in the art and sequence alignments of coronaviruses have been reported previously (see, e.g., Walls et al. Nature 2016 531:114-117 in particular Extended Data FIG. 9).

Seven strains of coronaviruses are known to infect humans. Infection by four of the human coronaviruses (“common coronaviruses”), i.e., HCoVs-229E, OC43, NL63, and HKU1 infections typically result in mild to severe upper and lower respiratory tract disease. These viruses account for approximately 15% of common colds. Infection by three of the human coronaviruses, i.e., Middle East respiratory syndrome-related coronavirus (MERS-CoV), Severe acute respiratory syndrome coronavirus (SARS-CoV), and Severe acute respiratory syndrome coronavirus 2 (SARS-CoV-2); can lead to severe symptoms as well as death. Humans likely acquired MERS-CoV from dromedary camels; SARS-CoV from bats; and bats may also have been the reservoir host for SARS-CoV-2.

Viral receptor engagement and viral fusion are essential for virus entry in a host cell. The spike protein of coronaviruses binds to different targets to mediate infectivity. SARS-CoV-2, SARS-CoV-1 and NL63 bind to ACE2, OC43 and HKU1 bind to 9-O-acetylated sialic acid, MERS-CoV binds to DPP4 and sialic acid, and 229E binds to APN. A further differentiating factor between these viruses is the presence or absence in the viral spike protein S of a human furin cleavage site. It is present in the S protein of SARS-CoV-2, OC43, HKU1, and MERS-CoV, but absent from the S protein of NL43, 229E and SARS-CoV.

SARS-CoV-2 is also referred to as COVID-19 virus (i.e., the novel coronavirus that causes coronavirus disease 2019). The Covid-19 pandemic has resulted in an enormous health crisis for which novel solutions are urgently needed to prevent, ameroliate or cure this infection. One object on the present disclosure is to provide methods and compositions for increasing immunity against SARS-CoV-2, as well as other pathogenic coronaviruses.

The major risk group for severe COVID-19 resulting in hospitalization peaks at ages between 70 and 80 and COVID-19 mortality peaks between 80 and 90 in countries like the Netherlands. This group has increasing numbers of comorbidities of which the majority is non-communicable. COVID-19 is thus an emergent disease of the aging, like pneumococcal pneumonia, severe influenza, shingles and pertussis (Santesmasses D et al. COVID-19 is an emergent disease of the aging, MedRxiv 2020). One object on the present disclosure is to provide methods and compositions for generating herd immunity in the general population in order to protect individuals that (because of age or otherwise) do not respond well to vaccination.

### SUMMARY OF THE INVENTION

The disclosure provides peptide and nucleic acid-based vaccines for increasing the immune response against multiple coronaviruses. The disclosure provides the following preferred embodiments. However, the invention is not limited to these embodiments.

In one aspect the disclosure provides a pharmaceutical composition for use in increasing immunity in a human individual against at least two different coronaviruses, preferably wherein at least one of the coronaviruses is selected from SARS-CoV-1, MERS-CoV and SARS-CoV-2, said composition comprising a peptide, or a nucleic acid molecule encoding the peptide, said peptide comprising at least a part of the S2 ectodomain of the S (spike) protein from at least one human coronaviruses (HCoV) selected from HCoV-NL63, HCoV-OC43, HCoV-229E and HCoV-HKU1, wherein said composition is administered intranasally.

In one aspect the disclosure provides a pharmaceutical composition for increasing immunity in a human individual against a highly pathogenic coronavirus, said composition comprising a peptide, or a nucleic acid molecule encoding the peptide, said peptide comprising at least a part of the S2 ectodomain of the S (spike) protein from at least one human coronaviruses (HCoV) selected from HCoV-NL63, HCoV-0C43, HCoV-229E and HCoV-HKU1, preferably HCoV-NL63.

In some embodiments, the composition further comprises a second peptide, or a nucleic acid molecule encoding the peptide, said peptide comprising at least a part of the S2 ectodomain of the S (spike) protein from at least one human coronaviruses (HCoV) selected from HCoV-NL63, HCoV-OC43, HCoV-229E and HCoV-HKU1 and/or a peptide, or a nucleic acid molecule encoding the peptide, said peptide comprising to at least a part of the S2 ectodomain of the S (spike) protein from at least one highly pathogenic human coronavirus selected from SARS-CoV-1, MERS-CoV and SARS-CoV-2 or from an animal coronavirus.

Preferably, the peptide comprises the fusion peptide, the HR1 heptad repeat, or the HR2 heptad repeat of the S protein.

Preferably, the peptide is conjugated to an immune stimulant, preferably wherein the immune stimulant is selected from Keyhole Limpet Hemocyanin (KLH), Concholepas Concholepas Hemocyanin (CCH), Bovine Serum Albumin (BSA), or Ovalbumin (OVA).

Preferably, wherein the peptide, or a nucleic acid molecule encoding the peptide, is provided in or attached to a nanoparticle.

Preferably, the nucleic acid molecule is comprised in a vector, preferably a viral vector.

Preferably, the composition comprises an adjuvant, preferably wherein the adjuvant is selected from a TLR3 or TLR 4 agonist, murabutide, betaglycan, and/or cholera toxin.

Preferably, the composition is formulated for intranasal delivery.

Preferably, the composition increases immunity against at least two different coronaviruses, preferably wherein at least one of the coronaviruses is selected from SARS-CoV-1, MERS-CoV and SARS-CoV-2.

In one aspect, the disclosure also provided methods of treatment and the use of the pharmaceutical composition for increasing immunity in a human individual against one or more highly pathogenic coronaviruses.

Preferably, the composition is administered intranasally.

Preferably, the human is not considered to be at risk for COVID-19 severe illness. Preferably, the individual is administered a booster dose to maintain protective immunity.

Preferably, the methods of treatment and uses further comprise subsequently testing the immunity of said individual against a pathogenic coronavirus.

### DETAILED DESCRIPTION OF THE DISCLOSED EMBODIMENTS

Typical vaccine development relies on using the disease-causing virus and either attenuating the virus for use as a ‘live attenuated vaccine’ or inactivating the virus. The solution provided by the approach described herein does not rely on using the disease-causing virus to develop a vaccine. While not wishing to be bound by theory, it is proposed herein that vaccination with a peptide comprising at least a part of the S2 ectodomain of the S (spike) protein from a first “common” coronavirus induces a cross-reactive immune response against a second coronavirus, in particular a highly pathogenic coronavirus. The resulting cross-reactive immune response increases the immunity in an individual against other coronaviruses. The disclosure further contemplates the use of nucleic acid-based vaccines comprising a nucleic acid encoding at least a part of the S2 ectodomain of the S (spike) protein from a first “common” coronavirus.

As a response to the SARS-CoV-2 pandemic, various efforts have been made to develop a SARS-CoV-2 specific vaccine in order to prevent infection. While not wishing to be bound by theory, it is proposed herein that vaccines developed based on SARS-CoV-2 specific sequences are less effective at targeting evolving SARS-CoV-2 strains, other pathogenic coronaviruses, or animal coronaviruses susceptible to cross-species transmission. As demonstrated by the MERS-CoV, SARS-CoV-1, and SARS-CoV-2 outbreaks, cross-species transmission of coronavirus leads to disease emergence. Vaccines that induce highly specific responses to a particular HCoV strain are unlikely to provide significant, if any, protection against such newly emergent HCoVs.

The disclosure provides methods and compositions for vaccinating individuals against multiple strains of coronavirus. In particular the methods and compositions are for increasing immunity in an individual against multiple strains of coronavirus. As used herein, the pharmaceutical compositions comprising peptides or nucleic acid molecules (including vectors) may also be referred to as vaccines.

The term “increasing immunity” refers to increasing an individual's immune response against a particular antigen (e.g., coronavirus). Increased immunity can lead to increased resistance to infection or may improve an individual's ability to fight infection (e.g., infection may be cleared before symptoms arise or symptoms experienced are milder). Increased immunity does not require full immunity, but also includes partial immunity. As will be clear to a skilled person, the methods and compositions disclosed herein may be used to prevent or reduce coronaviral infection and/or reduce the severity of coronaviral infection. Said methods and compositions may also be used to prevent or reduce the severity of symptoms associated with coronaviral infection.

Increased immunity can include an increase in innate immunity and/or adaptive immunity. As is known to a skilled person, the innate immune response is immediate (generally between 0-96 hours) and is considered the “first line of defence” against non-self pathogens. The innate immune response in generally mediated by natural killer cells, macrophages, neutrophils, dendritic cells, mast cells, basophils, and eosinophils. Natural killer cells, e.g., can target and destroy infected cells. Pattern recognition receptors (such as Toll-like receptors, nucleotide-binding oligomerization domain-like receptors, and retinoic acid inducible gene-I-like receptors) can detect specific viral components such as viral RNA or DNA in order to induce interferons and other pro-inflammatory cytokines. Interferons type I and type III are considered the main effectors of the antiviral immune response and activate “interferon-stimulated genes”. Retinoic acid inducible gene-I-like receptors can recognise viral RNA in the cytoplasm of infected cells and Toll-like receptors (e.g., TLR3, TLR7, TLR8, and TLR9) can detect viral RNA or DNA in endosomal compartments in immune cells and at the cell surface (e.g., TLR4). For a review of Toll-like receptor function in innate immunity against viral infection, see, e.g., Uematsu and Akira JBC 2007 282:15319-15232. TLR3, for example, is activated by double-stranded RNA and upon activation induces the transcription factors: activator protein 1 (AP-1), nuclear factor kappa-light-chain-enhancer of activated B cells (NF-κB) and interferon regulator factors 3 and 7 (IRF3 and IRF7).

In contrast, the adaptive immune response (i.e., acquired immunity) is not immediate but is long lasting and is generally mediated by T-cells and B-cells. Cellular immunity is mediated by T-cells and is generally directed at infected cells. Humoral immunity relies on B-cells to produce antibodies against a pathogen specific antigen and is generally directed at freely circulating pathogens or those outside infected cells. The adaptive immune response is specific to the particular pathogen. However, in some cases errors can occur and the adaptive immune response may attach self-antigens which can lead to the development of autoimmune disease.

In preferred embodiments, increased immunity refers to an increase in innate immunity. Methods for confirming an increase in innate immunity are known in the art and include, e.g., determining the total number of leukocytes and of the subclasses of white cells (e.g., neutrophils, monocytes, lymphocytes, T lymphocytes, B lymphocytes, CD4+ cells, CD8+ cells, natural killer cells) in the circulation, for example using immunological staining procedures and flow cytometry.

In some embodiments, increased immunity refers to an increase in T-cell mediated immunity. The induction and/or enhancement of the T-cell immune response in an individual can be detected using methods known in the art. For example, an increased number of coronavirus-specific T-cells can be measured. Methods and techniques for the characterization of virus-specific CD4+ T cells and CD8+ T cells are known in the art. Typically, responsive T cells secrete one or more specific cytokines upon exposure to viral antigens which can be detected, for example, using intracellular cytokine staining assays.

In some embodiments, increased immunity refers to an increase in antibody mediated immunity (i.e., humoral immune response). Induction and/or enhancement of the humoral immune response may involve the activity of B cells. This may be reflected by an increased frequency of peripheral blood B lymphocytes capable of differentiation into antibody-secreting plasma cells upon encounter with coronavirus antigens. The antibody mediated response may also result in the production of coronavirus antibodies (e.g. IgA and IgG). A secretory IgA immune response at one or more mucosal sites can be particularly advantageous since this can assist in the neutralisation of coronavirus upon entry through mucosal membranes. Methods for detecting B-cells and antibodies that bind the coronavirus are known to the skilled person and include flow cytometry and immunohistochemistry.

In some embodiments, increased immunity refers to providing sterilizing immunity. In contrast to immunity that allows for infection but is effective at clearing the infection, sterilizing immunity prevents an effective viral infection. In some embodiments, the compositions disclosed herein provide sterilizing immunity.

In preferred embodiments, increased immunity refers to increasing (or boosting) pre-existing immunity. Most individuals have previously been infected with one or more common human coronaviruses. While not wishing to be bound by theory, such individuals likely have memory B-cells that encode antibodies which recognize human coronaviruses and in some instances such antibodies may be cross-reactive against a number of different coronavirus strains. In some embodiments, the compositions provided herein stimulate such B-cells resulting in the production of cross-reactive coronavirus antibodies.

The disclosure provides methods and compositions for increasing immunity against a coronavirus in a human. The compositions are cross-protective (i.e., increase immunity) against two or more different coronaviruses. Preferably, the peptides, or nucleic acid molecules encoding said peptides, disclosed herein are cross-protective (i.e., increase immunity) against two or more different coronaviruses. Preferably, at least one or at least two of the coronaviruses is a highly pathogenic virus, or rather a virus which can lead to severe symptoms in infected patients. In some embodiments, a highly pathogenic virus as used herein refers to a virus having a fatality rate of 1% are higher. Exemplary highly pathogenic coronaviruses include MERS-CoV (fatality rate of around 34%), SARS-CoV-1 (fatality rate of around 9.5%), and SARS-CoV-2 (fatality rate of around 2%) (Petrosillo et al. Clinical Microbiology and Infection Volume 26, Issue 6, June 2020, Pages 729-734). Virulence can also be defined based on the severity of symptoms. For example, in some embodiments, a highly pathogenic coronavirus as used herein refers to a virus that causes acute respiratory distress syndrome in at least 10% of infected individuals, which includes SARS-CoV-1, SARS-CoV-2, and MERS-CoV (Petrosillo et al. 2020). In preferred embodiments, the coronavirus is an alpha- or beta-coronavirus.

The disclosure provides pharmaceutical compositions comprising a peptide, or a nucleic acid molecule encoding said peptide, comprising at least a part of the S2 ectodomain of the S (spike) protein from at least one human coronaviruses (HCoV) selected from HCoV-NL63, HCoV-OC43, HCoV-229E and HCoV-HKU1. The source of coronavirus may be a clinical isolate, e.g., obtained from a nasal or throat swab of a human patient. The virus may be propagated on a cell line, for example a mammalian cell line, such as Calu-3, Vero cells, MadinDarby canine kidney (MDCK) cells, and PERC6 cells.

The sequences of “common” HCoVs are known in the art as are the sequences of the viral proteins encoded by said viruses. Sources of coronoavirus may be a clinical isolate, e.g., obtained from a nasal or throat swab of a human patient. The virus may be propagated on a cell line, for example a mammalian cell line, such as Calu-3, Vero cells, MadinDarby canine kidney (MDCK) cells, and PERC6 cells. Exemplary HCoV-NL63 sequences are described in WO2005017133. The genomic sequence of several clinical isolates are also publicly available; e.g., the genomic sequence of Human coronavirus NL63 isolate Amsterdam 496 is described in Pyrc et al. (J. Mol. Biol. 364 (5), 964-973 (2006) having accession number DQ445912 (VRL 21 Nov. 2006); the genomic sequence of Human coronavirus NL63 isolate Amsterdam 057 is described in Pyrc et al. (J. Mol. Biol. 364 (5), 964-973 (2006) having accession number DQ445911 (VRL 21 Nov. 2006); the genomic sequence of Human coronavirus NL63 isolate ChinaGD01 is described in Zhang et al. (Microbiol Resour Announc 9 (8), e01597-19 (2020)) having accession number MK334046 (28 Feb. 2020); the genomic sequence of Human coronavirus NL63 isolate ChinaGD05 is described in Zhang et al. (Microbiol Resour Announc 9 (8), e01597-19 (2020)) having accession number MK334045 (VRL 28 Feb. 2020); the genomic sequence of Human coronavirus NL63 isolate NL63/human/USA/891-4/1989 has accession number KF530114 (VRL 26 Sep. 2014); and the genomic sequence of Human coronavirus NL63 isolate NL63/human/USA/838-9/1983 has accession number KF530110 (VRL 26 Sep. 2014). A BLAST analysis of the six isolates listed above indicates that they share greater than 98% sequence identity.

The genomic sequence of several clinical isolates of HCoV-229E are publicly available; e.g., the genomic sequence of Human coronavirus 229E isolate 0349 is described in Farsani et al. (Virus Genes 45 (3), 433-439 (2012)) having accession number JX503060 (VRL 4 Apr. 2013); the genomic sequence of Human coronavirus 229E isolate J0304 is described in Farsani et al. (Virus Genes 45 (3), 433-439 (2012)) having accession number JX503061 (VRL 4 Apr. 2013); the genomic sequence of Human coronavirus 229E/Seattle/USA/SC9724/2018 has accession number MN369046 (VRL 21 Feb. 2020); the genomic sequence of Human coronavirus 229E/human/USA/933-40/1993 has accession number KF514433 (VRL 26 Sep. 2014); the genomic sequence of Human coronavirus 229E/BN1/GER/2015 has accession number KU291448 VRL (04-Sep.-2016); and the genomic sequence of Human coronavirus 229E/Seattle/USA/SC1212/2016 has accession number KY369911 (VRL 21 Feb. 2020). A BLAST analysis of the six isolates listed above indicates that they share greater than 99% sequence identity. In addition, the virus is also publicly accessible from ATCC as Human coronavirus 229E (ATCC VR-740; Hamre D, Procknow J J. A new virus isolated from the human respiratory tract. Proc. Soc. Exp. Biol. Med. 121: 190-193, 1966).

The genomic sequence of several clinical isolates of HCoV-HKU1 are publicly available; e.g., the genomic sequence of Human coronavirus HKU1 isolate Caen1 has accession number HM034837 (VRL 8 Oct. 2010); the genomic sequence of Human coronavirus HKU1 isolate genotype A has accession number AY597011 (VRL 27 Jan. 2006); the genomic sequence of Human coronavirus HKU1/human/USA/HKU1-15/2009 is described in Dominguez et al. (J. Gen. Virol. 95 (PT 4), 836-848 (2014)) having accession number KF686344 (VRL 26 Sep. 2014); the genomic sequence of Human coronavirus HKU1/human/USA/HKU1-5/2009 has accession number KF686340 (VRL 26 Sep. 2014); the genomic sequence of Human coronavirus HKU1/human/USA/HKU1-11/2009 has accession number KF430201 (VRL 26 Sep. 2014). A BLAST analysis of the six isolates listed above indicates that they share greater than 99% sequence identity.

The genomic sequence of several clinical isolates of HCoV-OC43 are publicly available; e.g., the genomic sequence of Human coronavirus OC43 isolate MDS16 has accession number MK303625 (VRL 30 Mar. 2019); the genomic sequence of Human coronavirus OC43 isolate MDS12 has accession number MK303623 (VRL 30 Mar. 2019); the genomic sequence of Human coronavirus OC43/Seattle/USA/SC9428/2018 has accession number MN310476 (VRL 21 Feb. 2020); the genomic sequence of Human coronavirus OC43/Seattle/USA/SC9430/2018 has accession number MN306053 (VRL 21 Feb. 2020); the genomic sequence of Human coronavirus OC43/human/USA/9211-43/1992 has accession number KF530097 (VRL 26 Sep. 2014); and the genomic sequence of Human coronavirus OC43/human/USA/873-6/1987 has accession number KF530087 (VRL 26 Sep. 2014). A BLAST analysis of the six isolates listed above indicates that they share greater than 98% sequence identity.

The spike proteins of the common human coronaviruses have been described and the S2 ectodomain proteins are also known. Peptides, or nucleic acid molecules encoding said peptides, are provided that comprise at least a part of to S2 ectodomain from HCoV-NL63, HCoV-OC43, HCoV-229E and HCoV-HKU1. In some embodiments, peptides corresponding to part of the S2 ectodomain from HCoV-NL63 are preferred. In some embodiments, peptides corresponding to part of the S2 ectodomain from HCoV-OC43 are preferred. In some embodiments, peptides corresponding to part of the S2 ectodomain from HCoV-229E are preferred. In some embodiments, peptides corresponding to part of the S2 ectodomain from HCoV-HKU1 are preferred.

As used herein the term peptide is interchangeable with the terms “polypeptide” and “protein”. The term “peptide” refers to short-chain molecules such as oligopeptides or oligomers or to long-chain molecules such as proteins. A peptide may also comprise modified amino acids. Thus, the peptides disclosed herein can also be modified by natural processes such as post-transcriptional modifications or by a chemical process. Thus, any modification of the peptide which does not have the effect of eliminating the immunogenicity of the peptide, is covered within the scope of the present disclosure. Preferably, peptides comprising between 5-50, more preferably between 12-20 amino acids of the S2 ectodomain are provided.

In addition to a peptide, or a nucleic acid molecule encoding said peptide, comprising at least a part of the S2 ectodomain of the S (spike) protein from at least one human coronaviruses (HCoV) selected from HCoV-NL63, HCoV-OC43, HCoV-229E and HCoV-HKU1, the composition may also comprise one or more peptides, or nucleic acid molecules encoding said peptides, comprising to at least a part of the S2 ectodomain of the S (spike) protein from at least one highly pathogenic human coronavirus selected from SARS-CoV-1, MERS-CoV and SARS-CoV-2 or from an animal (i.e., non-human) coronavirus. Exemplary non-human coronavirus known to a skilled person include, e.g., a swine, cattle, horses, camels, cats, dogs, rodents, birds, bats, rabbits, ferrets, or mink coronavirus.

Multiple peptides, or nucleic acid molecules encoding said peptides, may be included together in the composition. For example, multiple overlapping peptides from the S2 domain of a HCoV may be included. In some embodiments, the composition comprises at least two, at least three, at least four, at least five, or at least 10 different peptides. The peptides may comprise sequences from the same or different common coronaviruses. Several peptides may also be linked together. For example, multiple peptide sequences may be arranged as “beads-on-a-string”, such that the peptide sequences are directly linked together or may be linked through linker sequences. Amino acid sequences flanking or linking the peptides may comprise proteolytic cleavage sites. Various antigen presentation systems may be used such as, e.g., Epitope Antigen Presentation System (L.E.A.P.S.) from (Cel Sci).

The peptides, or nucleic acid molecules encoding said peptides, disclosed herein comprise at least part of the S2 ectodomain. Preferably, the peptides comprise or comprise at least a part of an UH domain (upstream helix), a fusion peptide, two heptad repeats (HR1 and HR2), a central helix, and a beta hairpin. More preferably, the peptides comprise the fusion peptide, the HR1 heptad repeat, or the HR2 heptad repeat of the S protein.

Preferably, the peptides, or nucleic acid molecules encoding said peptides, disclosed herein induce an immune response, or rather are immunogenic. Preferably, the peptides bind to an antibody or a T-cell receptor.

A peptide of the disclosure may comprise minor sequence variations as compared to the S2 ectodomain sequences, including, e.g., conservative amino acid substitutions. Conservative substitutions are well known in the art and refer to the substitution of one or more amino acids by similar amino acids. For example, a conservative substitution can be the substitution of an amino acid for another amino acid within the same general class (e.g., an acidic amino acid, a basic amino acid, or a neutral amino acid).

In some embodiments the composition further comprises an immune stimulant. Preferably, the peptide is conjugated to an immune stimulate. Exemplary immune stimulants include Keyhole Limpet Hemocyanin (KLH), Concholepas Concholepas Hemocyanin (CCH), Bovine Serum Albumin (BSA), and Ovalbumin (OVA).

In some embodiments the peptide is a synthetic peptide. The use of relatively short peptides is preferred for medical purposes as these can be synthesized in vitro efficiently. Chemical synthesis of peptides is routine practice and various suitable methods are known to the skilled person.

The peptides can also be produced using molecular genetic techniques, such as by inserting a nucleic acid into an expression vector, introducing the expression vector into a host cell, and expressing the peptide. Preferably, such peptides are isolated, or rather, substantially isolated from other polypeptides, cellular components, or impurities. The peptide can be isolated from other (poly)peptides as a result of solid phase protein synthesis, for example. Alternatively, the peptides can be substantially isolated from other proteins after cell lysis from recombinant production (e.g., using HPLC).

The disclosure further provides nucleic acid molecules encoding the peptides disclosed herein. Based on the genetic code, a skilled person can determine the nucleic acid sequences which encode the (poly)peptides disclosed herein. Based on the degeneracy of the genetic code, sixty-four codons may be used to encode twenty amino acids and translation termination signal.

In a preferred embodiment, the nucleic acid molecules are codon optimized. As is known to a skilled person, codon usage bias in different organisms can affect gene expression level. Various computational tools are available to the skilled person in order to optimize codon usage depending on which organism the desired nucleic acid will be expressed. Preferably, the nucleic acid molecules are optimized for expression in mammalian cells, preferably in human cells.

A further aspect of the disclosure provides vectors and expression vectors comprising the nucleic acid molecules disclosed herein. Preferred vectors are expression vectors.

It is within the purview of a skilled person to prepare suitable expression vectors for expressing the inhibitors disclosed hereon. An “expression vector” is generally a DNA element, often of circular structure, having the ability to replicate autonomously in a desired host cell, or to integrate into a host cell genome and also possessing certain well-known features which, for example, permit expression of a coding DNA inserted into the vector sequence at the proper site and in proper orientation. Such features can include, but are not limited to, one or more promoter sequences to direct transcription initiation of the coding DNA and other DNA elements such as enhancers, polyadenylation sites and the like, all as well known in the art. Suitable regulatory sequences including enhancers, promoters, translation initiation signals, and polyadenylation signals may be included. Additionally, depending on the host cell chosen and the vector employed, other sequences, such as an origin of replication, additional DNA restriction sites, enhancers, and sequences conferring inducibility of transcription may be incorporated into the expression vector. The expression vectors may also contain a selectable marker gene which facilitates the selection of host cells transformed or transfected. Examples of selectable marker genes are genes encoding a protein such as G418 and hygromycin which confer resistance to certain drugs, β-galactosidase, chloramphenicol acetyltransferase, and firefly luciferase. The expression vector may comprise one or more promoters suitable for the expression of the gene in, e.g., plant cells, fungal cells, bacterial cells, yeast cells, insect cells or other eukaryotic cells.

In some embodiments, the compositions comprise nucleic acid molecules encoding the peptides disclosed herein (i.e., nucleic acid-based vaccine). Both DNA and RNA molecules may be used. In some embodiments, the composition comprises nucleoside modified messenger RNA (mRNA) encoding at least a part of the S2 ectodomain of the S (spike) protein from at least one human coronaviruses (HCoV) selected from HCoV-NL63, HCoV-OC43, HCoV-229E and HCoV-HKU1. See Zhang et al. Front. Immunol., 27 Mar. 2019 for a review of mRNA vaccines.

The disclosure further provides vectors comprising the nucleic acids molecules disclosed herein. A “vector” is a recombinant nucleic acid construct, such as plasmid, phase genome, virus genome, cosmid, or artificial chromosome, to which another nucleic acid segment may be attached. The term “vector” includes both viral and non-viral means for introducing the nucleic acid into a cell in vitro, ex vivo or in vivo. The disclosure contemplates both DNA and RNA vectors. Vectors, including plasmid vectors, eukaryotic viral vectors and expression vectors are known to the skilled person. Vectors may be used to express a recombinant gene construct in eukaryotic cells depending on the preference and judgment of the skilled practitioner (see, for example, Sambrook et al., Chapter 16). For example, many viral vectors are known in the art including, for example, retroviruses, adeno-associated viruses, and adenoviruses. The methods for producing replication-deficient viral particles and for manipulating the viral genomes are well known. In some embodiments, the vaccine comprises an attenuated or inactivated viral vector comprising a nucleic acid disclosed herein.

In some embodiments, the peptide, or nucleic acid molecule encoding said peptide, is provided in or attached to a nanoparticle. Nanoparticle-based vaccines are known to a skilled person and are also described in Al-Halifa et al. Front. Immunol., 24 Jan. 2019. Nanoparticles are particles between 1 and typically 100 nanometers (nm) in size which can be used for, e.g., immobilising, protecting, and or presenting ligands. The peptides can thus be encapsulated in a nanoparticle or may be attached and exposed on the nanoparticle surface. A linker may be used to link one or more of the peptides disclosed herein to the nanoparticle. The linker may comprise, e.g., a sulphur-containing group, amino-containing group, phosphate-containing group or oxygen-containing group. Suitable linkers and encapsulation methods are known in the art.

Inorganic nanoparticles include carbon, silica, and metal-based particles. Suitable nanoparticles include carbon nanotubes (CNTs), carbon black nanoparticles, polystyrene nanoparticles, titanium dioxide (TiO2) nanoparticles, silicon dioxide (SiO2) nanoparticles, and aluminum oxyhydroxide nanoparticles. A preferred inorganic nanoparticle is a gold nanoparticle (AuNP).

In some embodiments, the nanoparticle is a polymeric nanoparticle such aspoly(d,l-lactide-co-glycolide) (PLG), poly(d,l-lactic-coglycolic acid)(PLGA), poly(g-glutamic acid) (g-PGA)m poly(ethylene glycol) (PEG), or polystyrene. The polymeric nanoparticle may comprise one or more natural polymers such as pullulan, alginate, inulin, or chitosan.

In some embodiments, the nanoparticle is a liposome. Liposomes are typically formed from biodegradable, non-toxic phospholipids and may comprise a self-assembling phospholipid bilayer shell with an aqueous core.

In some embodiments, the nanoparticle is a Self-assembling peptide nanoparticle (see Negandaripour et al. Biotechnology Advances 2017 35:575-596 for a review). The nanoparticle may be a self-assembling protein, e.g., comprising ferritin.

In some embodiments, the nanoparticle is an Immunostimulatory complexes (ISCOMs). “Classic” ISCOMs are prepared with saponin, cholesterol, phospholipid and amphipathic proteins. ISCOM Matrix has a similar structure but lacks the amphipathic proteins. Both forms are immunostimulatory and therefore act as vaccine adjuvants. See Bengtsson et al. (2011) ISCOM technology-based Matrix M™ adjuvant: success in future vaccines relies on formulation, Expert Review of Vaccines, 10:4, 401-403 for a review.

In some embodiments, the nanoparticles are virus-like particles (VLPs). Non-replicating VLPs resemble infectious virus particles in structure and morphology and contain immunologically relevant viral structural proteins.

Preferably, the compositions disclosed herein comprises a pharmaceutically acceptable excipient. The compositions may contain pharmaceutically acceptable auxiliary substances as required to approximate physiological conditions, such as pH adjusting and buffering agents, tonicity adjusting agents, wetting agents and the like.

Preferably, the compositions further comprise an adjuvant. As is known to a skilled person, adjuvants are used to enhance antigenicity. An adjuvant can include a suspension of minerals (e.g., alum, aluminum hydroxide, or phosphate) on which antigen is adsorbed; or water-in-oil emulsion, for example, in which antigen solution is emulsified in mineral oil (Freund incomplete adjuvant). Other suitable adjuvants include murabutide, cholera toxin, and betaglycan.

In some embodiments, the adjuvant is a TLR3 and/or TLR4 agonist. The agonist may be, e.g., a TLR ligand, a TLR mimic, or a small molecule. TLR3 agonists bind to an activate the toll-like receptor 3. Suitable TLR3 agonists are known to a skilled person and include double-stranded RNA (dsRNA) complex, for example polyribosinic:polyribocytidic acid (polyI:C) in particular Ampligen poly(I):poly(C12U) (Hemispherx Biopharma); polyadenosine-polyuridylic acid (poly A:U); rintatolimod (polyI: polyCU, Ampligen™); as well as polyionisinic-polycytidylic acid stabilized with poly-L-lysine and carboxymethylcellulose (Poly-ICLC). Further suitable TLR3 agonists include RGC100 (Naumann et al., Clin. Dev.Immunol. 283649, 2013), IPH-3102 (Basith et al., Exp. Opin. Ther. Pat.21: 927-944, 2011), CQ-07001 (Clinquest), IPH-31XX (Innate Pharma), and MCT-465-dsRNA (MultiCell Technologies).

Suitable TLR4 agonists are known to a skilled person and include bacterial lipopolysaccharide (LPS) or a variant thereof; monophosphoryl lipid A (MPL, MPLA, GLA, GLA-SE); AS15 or AS02b (Brichard et al., Vaccine 25(Suppl. 2):B61-B71, 2007; Kruit et al., J. Clin. Oncol. 26(Suppl): Abstract 9065, 2008); an aminoalkyl glucosaminide 4-phosphate (e.g., RC-529, E6020) or a variant thereof (Baldridge et al., J. EndotoxinRes. 8:453-458, 2002; Morefield et al., Clin. Vaccine Immunol. 14:1499-1504, 2007); picibanil (OK-432) (Hazim et al., Med. J. Malaysia 71(6):328-330, 2016); Spirulina complex polysaccharide (Kwanishi et al., Microbiol. Immunol. 57:63-73, 2013); ischitohexaose or a variant thereof (Panda et al., 8:e1002717, 2012; Barman et al., Cell Death Dis. 7:e2224, 2016); E5564 (Eritoran) Eisai); or CRX-675 or CRX-527 (GSK). Additional TLR4 agonists are described in US20150197527.

One aspect of the disclosure further provides a method for identifying a composition that increases immunity against a highly pathogenic virus. In particular the method identifies compositions that provide cross-reactive protection against at least two, preferably at least against three or more different coronaviruses. The method comprises preparing an alignment of the S2 sequences of the HCoVs-229E, OC43, NL63, HKU1, MERS-CoV, SARS-CoV-1, and SARS-CoV-2 and identifying regions of sequence similarity. Overlapping peptides (10-15 amino acids) from 229E, OC43, NL63, HKU1 from regions having high sequence similarity are generated. Individual peptides and peptide combinations are intranasally administered to rodents to identify peptides and peptide combinations that induce an immune response. The peptides may also be conjugated to various immune stimulants and/or provided in various nanoparticle systems.

The induced immune response may be determined by serological assays to detect antigen specific response. While not wishing to be bound by theory, vaccination with the peptides, or nucleic acid molecules encoding the peptides, is believed to induce the production of antibodies that bind to the respective S2 sequences. Western blot analysis, ELISA's, or any other known immunoassay may be used. For example, peptides may be linked to solid surfaces such as peptide microarrays (i.e., peptide chips). In some embodiments, Pepscan analysis can be performed, for example, where overlapping 15-mer linear peptides spanning the S2 domain are screened for immunoglobulin binding (see, e.g., Kramer et al. The Human Antibody Repertoire Specific for Rabies Virus Glycoprotein as Selected From Immune Libraries. Eur J Immunol. 2005 July;35(7):2131-45).

In vitro functional assays may also be used to detect relevant immune responses. For example, the plasma from vaccinated animals may be used in assays measuring viral fusion, replication, and/or replication. Viral fusion assays, infection assays, and replication assays are well-known to the skilled person and exemplary methods for performing such methods are described herein in the examples. For example, multiple cell-cell fusion assays that are mediated by the S protein of various HCoVs have been developed (Xia S, Yan L, Xu W, et al. A pan-coronavirus fusion inhibitor targeting the HR1 domain of human coronavirus spike. Sci Adv. 2019; 5(4)). Pseudotyped virus infection assays, such as those described in Lu et al. (Nat. Commun. 5, 3067 (2014), may also be used. Assays to measure HCoV replication have also been described (see, e.g., Brison, et al. J. Virol. 88, 1548-1563 (2014). As will be clear to a skilled person, complete inhibition is not required and a skilled person is able to identify peptides that upon vaccination significantly inhibit viral fusion, infection, or replication.

In some embodiments, assays, as described above, are performed to screen for a response against a panel of coronaviruses. In an exemplary embodiment of the disclosure, plasma (i.e., the antibodies present in the plasma) is screened to determine the effect on fusion, infection, or replication with HCoV-NL63, HCoV-OC43, HCoV-229E, HCoV-HKU1, SARS-CoV-1, MERS-CoV, SARS-CoV-2, and at least one animal coronavirus. Preferred peptides result in an immune response, such that the tested plasma inhibits fusion, infection, and/or replication of HCoV-NL63, HCoV-OC43, HCoV-229E, HCoV-HKU1, SARS-CoV-1, MERS-CoV, SARS-CoV-2, and at least one animal coronavirus. However, a skilled person will appreciate that peptides which induce a response to only a subset of coronaviruses are also useful.

Peptides and peptide combinations that induce an immune response will be further tested in in vivo challenge experiments. In vivo models of SARS-CoV-1, MERS-CoV and SARS-CoV-2 infection are known. Suitable in vivo models of SARS-CoV-2 infection are described in, e.g., Sia, S. F., Yan, L., Chin, A. W. H. et al. Pathogenesis and transmission of SARS-CoV-2 in golden hamsters. Nature (2020). Suitable in vivo models of MERS-CoV infection are described in, e.g., Kim J et al. Middle East Respiratory Syndrome-Coronavirus Infection Into Established hDPP4-Transgenic Mice Accelerates Lung Damage Via Activation of the Pro-Inflammatory Response and Pulmonary Fibrosis. J Microbiol Biotechnol. 2020 Mar. 28; 30(3):427-438. Suitable in vivo models of SARS-CoV-1 infection are described in, e.g., Roberts et al. Virus Research 2008 133:20-32.

The prevention or reduction of infection in vivo includes increased resistance to infection or an improved ability to fight infection (e.g., infection may be cleared before symptoms arise or symptoms experienced are milder). Mortality, weight loss, and lung pathology may be used as indicators of the ability to prevent or reduce infection in vivo. The candidate vaccines will be administered to test animals and subsequently challenged with SARS-CoV-1, MERS-CoV or SARS-CoV-2.

A further aspect of the disclosure provides methods of vaccinating individuals with the compositions disclosed herein. In one embodiment a method is provided for increasing immunity against one or more highly pathogenic coronaviruses, said method comprising administering the composition described herein to a human individual in need thereof. In one embodiment a method is provided for treating or preventing infection from a highly pathogenic coronaviruses, said method comprising administering to a human individual in need thereof the composition described herein.

Vaccines against some of the common coronaviruses have previously been described (see e.g., WO2005017133). The present compositions are useful for providing cross-reactive protection against at least two coronaviruses. In some embodiments, the vaccinated individual has previously been infected with one or more common viruses selected from HCoV-NL63, HCoV-OC43, HCoV-229E and HCoV-HKU1. In some embodiments, the composition comprises a peptide comprising at least a part of the S2 ectodomain of the S (spike) protein from HCoV-NL63 and said individual has previously been infected with HCoV-NL63. In some embodiments, the composition comprises a peptide comprising at least a part of the S2 ectodomain of the S (spike) protein from HCoV-OC43 and said individual has previously been infected with HCoV-OC43. In some embodiments, the composition comprises a peptide comprising at least a part of the S2 ectodomain of the S (spike) protein from HCoV-229E and said individual has previously been infected with HCoV-229E. In some embodiments, the composition comprises a peptide comprising at least a part of the S2 ectodomain of the S (spike) protein from HCoV-HKU1 and said individual has previously been infected with HCoV-HKU1.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. As will be clear to a skilled person, preventing infection does not require the absolute prevention of infection but includes reducing the severity of infection and/or the severity of symptoms associated with coronaviral infection.

Preferably, the compositions are administered locally, or rather not systemically. Local administration includes administration to the skin, eyes, and mucosal membranes.

In preferred embodiments, the compositions are applied to mucous membranes such as the bronchial, esophageal, nasal, and oral mucosa and the tongue. Preferably, the composition is administered intranasally. The composition may be applied to the lymphatic tissue of the nose in any convenient manner. However, it is preferred to apply it as a liquid stream or liquid droplets to the walls of the nasal passage. The intranasal composition can be formulated, for example, in liquid form as nose drops, spray, or suitable for inhalation, as powder, as cream, or as emulsion. In some embodiments, the compositions are provided as aerosol formulations (e.g., they can be “nebulized”) to be administered via inhalation. Aerosol formulations can be placed into pressurized acceptable propellants, such as dichlorodifluoromethane, propane, nitrogen, and the like.

As used herein, administration or administering in the context of treatment or prophylaxis of a subject is preferably in a “therapeutically effective amount”, this being sufficient to show benefit to the individual. Prescription of treatment, e.g. decisions on dosage etc., is within the responsibility of general practitioners and other medical doctors, and typically takes account of the condition of the individual patient, the site of delivery, the method of administration and other factors known to practitioners.

Preferably, the compositions are administered repeatedly or as a booster dose to maintain immunity. A boost vaccination may be administered, for example, about 1 month, 2 months, 4 months, 6 months, or 12 months after the initial vaccination, such that one exemplary vaccination regimen involves administration at 0, 0.5-2 and 4-8 months. In some embodiments, the compositions may be administered “as needed” before potential encounters with a highly pathogenic coronavirus.

In some embodiments, the individual treated is subsequently tested to determine whether immunity against a coronavirus has been induced. The efficacy of vaccination may be determined in a number of ways. For example, immunoreactivity to particular HCoVs may be determined, for example from plasma samples obtained from the vaccinated individuals. Methods for assaying immunoreactivity to HCoVs are known in the art. See, for example Chan K H et al. Serological Responses in Patients With Severe Acute Respiratory Syndrome Coronavirus Infection and Cross-Reactivity With Human Coronaviruses 229E, OC43, and NL63. Clin Diagn Lab Immunol. 2005 November; 12(11):1317-21; Kramer A R et al. The Human Antibody Repertoire Specific for Rabies Virus Glycoprotein as Selected From Immune Libraries. Eur J Immunol. 2005 July; 35(7):2131-45; and Pohl-Koppe 1995 Journal of Virological Methods 55:175-183. Such methods include, e.g., using viral proteins, or fragments thereof, in order to detect immunoglobulins present in plasma samples using, e.g., Western blot analysis, ELISA's, or any other known immunoassays. Preferably, the methods comprise determining (qualitatively or quantitatively) the presence of immunoglobulins that bind HCoV. As will be apparent to a skilled person, immunoreactivity or antibody binding to HCoV includes immunoreactivity or antibody binding to a protein encoded by said virus. Preferably, the methods comprise determining the immunoreactivity of the plasma samples to one or more HCoVs. Preferably, samples are tested for determining whether immunity has been induced against a highly pathogenic coronavirus. In addition, one may assay IL-17 levels (particularly IL-17A) by stimulating T cells derived from the individual after vaccination. The IL-17 levels may be compared to IL-17 levels in the same subject before vaccination. Increased IL-17 (e.g., IL-17A) levels would indicate a response to the composition.

As will be apparent to a skilled person, vaccination may not result in a detectable increased immunity in all vaccinated individuals. This may particularly be the case in the elderly. However, by vaccinating a significant proportion of the population, herd immunity is likely to develop.

As used herein, “to comprise” and its conjugations is used in its non-limiting sense to mean that items following the word are included, but items not specifically mentioned are not excluded. In addition, the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article. By way of example, “an element” means one element or more than one element.

The word “approximately” or “about” when used in association with a numerical value (approximately 10, about 10) preferably means that the value may be the given value of 10 more or less 1% of the value.

All patent and literature references cited in the present disclosure are hereby incorporated by reference in their entirety.

1. A method of increasing immunity in a human individual against at least two different coronaviruses, the method comprising:
administering intranasally to the human individual a composition comprising a peptide, or a nucleic acid molecule encoding the peptide, said peptide comprising at least a part of the S2 ectodomain of the S (spike) protein from at least one human coronaviruses (HCoV) selected from HCoV-NL63, HCoV-OC43, HCoV-229E and HCoV-HKU1.

2. A pharmaceutical composition for increasing immunity in a human individual against a highly pathogenic coronavirus, said composition comprising a peptide and/or a nucleic acid molecule encoding the peptide, said peptide comprising at least a part of the S2 ectodomain of the S (spike) protein from at least one human coronaviruses (HCoV) selected from HCoV-NL63, HCoV-OC43, HCoV-229E and HCoV-HKU1.

3. The composition of claim 2, further comprising a second peptide, and/or a nucleic acid molecule encoding the second peptide, said second peptide comprising at least a part of the S2 ectodomain of the S (spike) protein from at least one human coronaviruses (HCoV) selected from HCoV-NL63, HCoV-OC43, HCoV-229E and HCoV-HKU1 and/or
a third peptide, and/or a nucleic acid molecule encoding the third peptide, said third peptide comprising to at least a part of the S2 ectodomain of the S (spike) protein from at least one highly pathogenic human coronavirus selected from SARS-CoV-1, MERS-CoV and SARS-CoV-2 or from an animal coronavirus.

4. The composition of claim 2, wherein the peptide comprises the fusion peptide, the HR1 heptad repeat, or the HR2 heptad repeat of the S protein.

5. The composition of claim 1, wherein the peptide is conjugated to an immune stimulant.

6. The composition of claim 2, wherein the peptide, or a nucleic acid molecule encoding the peptide, is in or attached to a nanoparticle.

7. The composition of claim 2, wherein the nucleic acid molecule is comprised in a vector.

8. The composition of claim 2, comprising an adjuvant.

9. The composition of claim 2, wherein the composition is formulated for intranasal delivery.

10. The composition of claim 2, wherein the composition increases immunity against at least two different coronaviruses.

11. A method for increasing immunity in a human individual against one or more highly pathogenic coronaviruses, the method comprising administering to the human individual the composition of claim 2.

12. The method of claim 11, wherein the composition is administered intranasally.

13. The method according to claim 11, wherein the human individual is not considered to be at risk for COVID-19 severe illness.

14. The method according to claim 11, further comprising administering to the human individual a booster dose to maintain protective immunity.

15. The composition for use method according to claim 11, further comprising, subsequent to the administration, testing the immunity of said human individual against a pathogenic coronavirus.

16. The method according to claim 1, wherein at least one of the coronaviruses is selected from SARS-CoV-1, MERS-CoV and SARS-CoV-2.

17. The composition of claim 5, wherein the immune stimulant is selected from Keyhole Limpet Hemocyanin (KLH), Concholepas Concholepas Hemocyanin (CCH), Bovine Serum Albumin (BSA), and Ovalbumin (OVA).

18. The composition of claim 8, wherein the adjuvant is selected from a TLR3 or TLR 4 agonist, murabutide, betaglycan, and/or cholera toxin.

19. The composition of claim 10, wherein at least one of the at least two different coronaviruses is selected from SARS-CoV-1, MERS-CoV and SARS-CoV-2.
